(19) United States
(12) Patent Application Publication
(10) Pub. No.: US 2022/0243286 A1
(43) Pub. Date: Aug. 4, 2022

(54) MOLECULAR MARKER, GENE OF MAIZE EAR ROT RESISTANCE AND USE THEREOF

(71) Applicant: HENAN AGRICULTURAL UNIVERSITY, Zhengzhou City (CN)

(72) Inventors: Zijian ZHOU, Zhengzhou City (CN); Jianyu WU, Zhengzhou City (CN); Jiafa CHEN, Zhengzhou City (CN); Xinyi CHEN, Zhengzhou City (CN); Xulu HAN, Zhengzhou City (CN); Xingchi GUO, Zhengzhou City (CN); Peiyuan TENG, Zhengzhou City (CN)

(21) Appl. No.: 17/579,984

(22) Filed: Jan. 20, 2022

(30) Foreign Application Priority Data
Feb. 4, 2021 (CN) 202110153447.2

(51) Int. Cl. C12Q 1/6895

(52) U.S. Cl. CPC C12Q 1/6895; C12Q 2600/172; C12Q 2600/13; C12Q 2600/156

(57) ABSTRACT

The disclosure belongs to the technical field of functional molecular marker, and discloses a molecular marker of maize ear rot resistance, gene and use thereof. In the disclosure a major gene zmSRR1 of maize ear rot resistance is cloned, and the gene's resistance to Fusarium verticillioides is checked by transgenic method. By comparing sequences and a candidate gene association analysis combined with resistance phenotypes, three natural variation sites affecting maize ear rot resistance are confirmed, and are found to be combined into five haplotypes in natural materials. Among which one is a high ear rot susceptible haplotype, so that the steps of detecting variation sites, confirming haplotypes, identifying types of resistance genes in maize, confirming whether disease resistance improvement can be targeted, tracking and monitoring the subsequent breeding are conducted.

This patent application claims the benefit and priority of Chinese Patent Application No. 202110153447.2 filed on Feb. 4, 2021, the disclosure of which is incorporated by reference herein in its entirety as part of the present application.

### TECHNICAL FIELD

The present disclosure belongs to the technical field of functional molecular marker, in particular relates to a molecular marker, gene and use thereof.

### BACKGROUND ART

Currently, maize ear rot is a major disease worldwide, which affects the yield, as well as produces toxins, leading to poisoning and carcinogenicity of human and livestock. In recent years, with changes of climate and production mode, the incidence of maize ear rot become seriously over time, resulting in great affects of the food production safety. A main pathogen of ear rot is soil-borne fungi of Fusarium with huge difficulties in a conventional chemical control. Thus, breeding disease-resistant maize varieties is a best way to solve this problem, as ear rot susceptible materials are one-vote veto during new maize cultivar certification by the state. Ear rot resistance is a minor gene controlled quantitative trait, and a traditional breeding method based on phenotypic screening is inefficient and costly. However, genes and functional markers related to ear rot resistance are not yet reported, so that molecular breeding can not replace traditional breeding methods.

By analysis described above, the problems and defects of the prior art are as follows: for ear rot, conventional chemical control and prevention are extremely difficult, and a traditional breeding method based on phenotypic screening is inefficient and costly.

The difficulty to solve the above problems and defects is that no resistance genes and functional markers were found and effective molecular breeding could not be carried out.

A significance of solving the above problems and defects is to open up a possibility of molecular breeding of ear rot resistance, reduce the cost of breeding and improve breeding efficiency, by cloning the major genes of ear rot resistance and developing functional molecular markers.

### SUMMARY

Aiming at the problems existing in the prior art, the present disclosure provides a molecular marker, gene of maize ear rot resistance and use thereof.

The present disclosure is realized in this way, a molecular marker of maize ear rot resistance, wherein the molecular marker comprises three variation sites of srr1-1, srr1-2, and srr1-3, respectively for a 34^{th} base A/G mutation, a 1915^{th} A/G mutation, and a 2033^{th} G/T mutation of the zmSRR1 gene sequence.

Another objective of the present disclosure is to provide a gene to verify the molecular marker of ear rot resistance, wherein the gene is zmSRR1.

Furthermore, the gene is cloned from the ear rot resistance gene.

Another objective of the present disclosure is to provide a method for developing functional molecular markers, wherein the molecular marker of maize ear rot resistance is used in the method.

Another objective of the present disclosure is to provide a type detection method of a disease resistance gene of maize materials, wherein the molecular marker of maize ear rot resistance is used in the type detection method.

Another objective of the present disclosure is to provide a maize molecular breeding method, wherein the molecular marker of maize ear rot resistance is used in the maize molecular breeding method.

Combined with all technical solutions above, the advantages and positive effects of the present disclosure are as follows: a molecular marker of maize ear rot resistance, gene and use thereof herein can effectively improve the selection efficiency of disease resistance sites and reduce the time and cost due to a large number of field tests. The present disclosure can effectively improve the efficiency of disease resistance sites selection, reduce the time and cost of a large number of field tests, and improve the efficiency of breeding methods for continuing phenotypic screening, which may be used for detection of maize materials or molecular breeding.

### BRIEF DESCRIPTION OF THE DRAWINGS

In order to make technical solutions of embodiments herein more clearly, briefly introduction of drawings used in the embodiments of the present application is shown as follows. Obviously, drawings described below are only some embodiments of the present application. Other drawings can be obtained from the drawings herein without creative work by those skilled in the art.

FIG. 1 is a flow chart of a method of using a molecular marker of maize ear rot resistance shown in an embodiment of the present disclosure.

### DETAILED DESCRIPTION OF THE EMBODIMENTS

In order to make objectives, technical solutions and advantages herein more clearly, combined with the embodiments below, the disclosure is further described in detail. It should be understood that the specific embodiments described here are only used to explain the disclosure, not to limit it.

In view of the problems in the prior art, the present disclosure provides molecular marker of maize ear rot resistance, gene and use thereof. The present disclosure is described in detail below with reference to the accompanying drawings.

The molecular markers of maize ear rot resistance of the present disclosure are three variant sites of srr1-1, srr1-2, srr1-3, respectively for the 34^{th} base A/G mutation, the 1915^{th} A/G mutation, and the 2033^{th} G/T mutation in the zmSRR1 gene sequence.

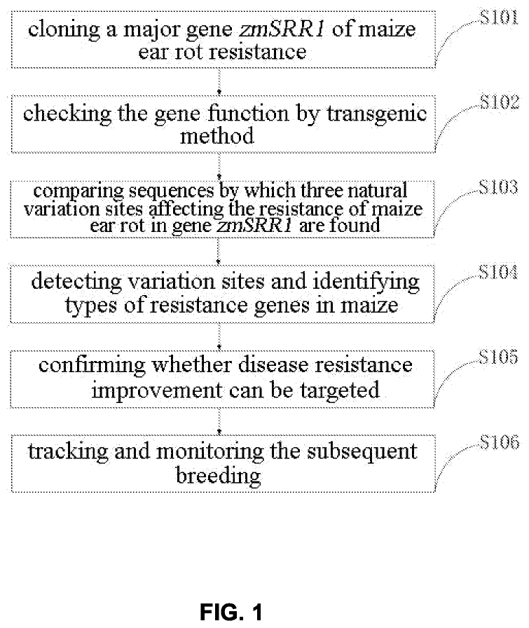

As shown in FIG. 1, a method of using the molecular marker of maize ear rot resistance provided by an embodiment of the present disclosure comprises:

S101, cloning a major gene zmSRR1 of maize ear rot resistance;

S102, checking the gene function by transgenic method;

S103, comparing sequences by which three natural variation sites affecting the resistance of maize ear rot in gene zmSRR1 are found;

S104, detecting variation sites and identifying types of resistance genes in maize;

S105, confirming whether disease resistance improvement can be targeted by association the marker results and phenotype of populations;

S106, tracking and monitoring the subsequent breeding.

A major gene zmSRR1 of maize ear rot resistance is cloned in present disclosure (also known as GRMZM2G009818, Zm00001d027645, LOC103631713, MDIS1-interacting receptor like kinase 1), derived from Ming Ju #, Zijian Zhou #, Cong Mu, Xuecai Zhang, Jingyang Gao, Yakun Liang, JiafaChen, Yabin Wu, Xiaopeng Li, Shiwei Wang, Jingjing Wen, Luming Yang*, Jianyu Wu*, Dissecting the genetic architecture of Fusarium verticillioides seed rotresistance in maize by combining QTL mapping and genome-wide associationanalysis, Scientific Reports, 2017, 7(1).

TABLE 1
Statistical analysis of 3 functional markers and their
haplotypes on the resistance phenotype of maize ear rot

| | | statistics of incidence grade of ear rot | | | |
|---|---|---|---|---|---|
| | | | mean and | | |
| | | | significant | | phenotypic |
| | | number of | of | | contribution |
| marker | genotype | materials | difference | significant | rate |
| srrl-1 | A | 39 | 2.87A | 4.68E−05 | 12.0% |
| | G | 93 | 2.35B | | |
| srrl-2 | A | 76 | 2.70A | 9.61E−04 | 7.5% |
| | G | 66 | 2.32B | | |
| srrl-3 | G | 85 | 2.69A | 6.25E−04 | 8.0% |
| | T | 57 | 2.29B | | |
| haplotype | A + | 29 | 3.03a | 3.89E−05 | 18.1% |
| | A + G | | | | |
| | A + | 10 | 2.40b | | |
| | G + T | | | | |
| | G + | 41 | 2.40b | | |
| | A + G | | | | |
| | G + | 9 | 2.55b | | |
| | G + G | | | | |
| | G + | 43 | 2.27b | | |
| | G + T | | | | |

Through detection of markers and identification of disease resistance in 142 maize inbred lines, three functional markers are found to be combined into five haplotypes, and contribute to 18.1% of the phenotypic variation, wherein, A+A+G is a high ear rot susceptible haplotype.

TABLE 2
Numbers of materials and proportions of different haplotypes of SRR1 gene in
different maize breeding kin materials detected based on 3 functional markers

| | | | | | | Total (numbers |
|---|---|---|---|---|---|---|
| kin | G + G + G | G + G + T | A + G + T | G + A + G | A + A + G | of materials) |
| CML | 3(4.2%) | 28(38.9%) | 10(13.9%) | 24(33.3%) | 7(9.7%) | 72 |
| Reid | 0(0%) | 10(76.9%) | 0(0%) | 2(15.4%) | 1(7.7%) | 13 |
| Lan | 1(20%) | 3(60%) | 0(0%) | 1(20%) | 0(0%) | 5 |
| P | 1(6.3%) | 2(12.5%) | 0(0%) | 11(68.8%) | 2(12.5%) | 16 |
| TSPT | 3(13.6%) | 1(4.5%) | 0(0%) | 0(0%) | 18(81.8%) | 22 |

Through marker detections of maize inbred lines with different breeding kin, the high ear rot susceptible haplotypes were found concentrated in a kin material with Tangsipingtou (TSPT). Tangsipingtou kin inbred line is the main male parent type of maize planting area in Huang-Huai-Hai, China, with excellent traits in breeding such as early maturity and pollen dispersal, but poor resistance to ear rot. Marker detections show that the proportion of SRR1 gene with susceptible genotypes in Tangsipingtou kin materials is up to 81.8%. Therefore, the molecular markers of ear rot resistance can effectively improve the resistance of most Tangsipingtou kin inbred lines to ear rot and promote the breeding of disease-resistant maize varieties.

The above are only specific embodiments of the present disclosure, and the protection scope of the disclosure is not limited to this. Within the technical scope disclosed herein, any modifications, equivalent replacements, improvements, etc. made within the spirit and principles of the present disclosure shall be covered by the claimed scope of the present disclosure.

1. A method for developing a functional molecular marker, wherein the method uses a molecular marker of maize ear rot resistance, the molecular marker comprises three variation sites of srr1-1, srr1-2, and srr1-3, respectively for a 34^{th} base A/G mutation, a 1915^{th} A/G mutation, and a 2033^{th} G/T mutation of the zmSRR1 gene sequence.

2.-4. (canceled)

5. A type detection method of a disease resistance gene of maize materials, wherein the method uses a molecular marker of maize ear rot resistance, the molecular marker comprises three variation sites srr1-1, srr1-2, and srr1-3, respectively for a 34^{th} base A/G mutation, a 1915^{th} A/G mutation, and a 2033^{th} G/T mutation of the zmSRR1 gene sequence.

6. A maize molecular breeding method, wherein the method uses a molecular marker of maize ear rot resistance, molecular marker comprises three variation sites of srr1-1, srr1-2, and srr1-3, respectively for a 34^{th} base A/G mutation, a 1915^{th} A/G mutation, and a 2033^{th} G/T mutation of the zmSRR1 gene sequence.
